SEQUENCE LISTING (65 sequences)

SEQ ID NO: 1 - DNA, 20 bp, Artificial Sequence
  Designd Tag referred to as “Tag 1” in paragraph [0054]
tcccgcgaaattaatacgac

SEQ ID NO: 2 - DNA, 18 bp, Artificial Sequence
  Designd Tag referred to as “Tag 2” in paragraph [0054]
gctggagctctgcagcta

SEQ ID NO: 3 - DNA, 39 bp, Artificial Sequence
  Designd primer referred to as “primer 1” in paragraph [0054]
tcccgcgaaattaatacgacgcatctgcctcacctccac

SEQ ID NO: 4 - DNA, 38 bp, Artificial Sequence
  Designd primer referred to as “primer 2” in paragraph [0054]
gctggagctctgcagctatgcctccttctgcatggtat

SEQ ID NO: 5 - DNA, 20 bp, Artificial Sequence
  Designd probe referred to as “dual biotinylated probe” in paragraph [0056]
tcccgcgaaattaatacgac

SEQ ID NO: 6 - DNA, 20 bp, Artificial Sequence
  Designd primer referred to as “forward primer” in paragraph [0059]
tcccgcgaaattaatacgac

SEQ ID NO: 7 - DNA, 18 bp, Artificial Sequence
  Designd primer referred to as “reverse primer” in paragraph [0059]
gctggagctctgcagcta

SEQ ID NO: 8 - DNA, 20 bp, Artificial Sequence
  Designd primer referred to as “SBE primer” in paragraph [0064]
agccgaagggcatgagctgc

SEQ ID NO: 9 - DNA, 17 bp, Artificial Sequence
  Designd probe referred to as “ASH probe” in paragraph [0065]
atgagctgcatgatgag

SEQ ID NO: 10 - DNA, 16 bp, Artificial Sequence
  Designd probe referred to as “wild-type ASH probe” in paragraph [0065]
tgagctgcgtgatgag

SEQ ID NO: 11 - DNA, 17 bp, Artificial Sequence
  Designd probe referred to as “biotinylated probe” in paragraph [0065]
cggacatagtccaggag

SEQ ID NO: 12 - DNA, 34 bp, Artificial Sequence
  Designd probe referred to as “LNA fullmatch Alexa 647” in paragraph [0070]
atgagctgcatgatgagtactcgacgcactactc

SEQ ID NO: 13 - DNA, 33 bp, Artificial Sequence
  Designd probe referred to as “LNA fullmatch Alexa 488” in paragraph [0070]
tgagctgcgtgatgagtactcgacgcactactc

SEQ ID NO: 14 - DNA, 34 bp, Artificial Sequence; identical to SEQ ID NO: 12
  Designd probe referred to as “DNA fullmatch Alexa 647” in paragraph [0070]

SEQ ID NO: 15 - DNA, 33 bp, Artificial Sequence; identical to SEQ ID NO: 13
  Designd probe referred to as “DNA fullmatch Alexa 488” in paragraph [0070]

SEQ ID NO: 16 - DNA, 20 bp, Artificial Sequence
  Designd primer referred to as “Tag 1” in Fig.10
tcccgcgaaattaatacgac

SEQ ID NO: 17 - DNA, 18 bp, Artificial Sequence
  Designd primer referred to as “Tag 2” in Fig.10
gctggagctctgcagcta

SEQ ID NO: 18 - DNA, 43 bp, Artificial Sequence
  Designd primer referred to as “Tag119del” in Fig.10
tcccgcgaaattaatacgacaagttaaaattcccgtcgctatc

SEQ ID NO: 19 - DNA, 36 bp, Artificial Sequence
  Designd primer referred to as “Tag219del” in Fig.10
gctggagctctgcagctagacccccacacagcaaag

SEQ ID NO: 20 - DNA, 39 bp, Artificial Sequence; identical to SEQ ID NO: 3
  Designd primer referred to as “Tag1T790M” in Fig.10

SEQ ID NO: 21 - DNA, 38 bp, Artificial Sequence
  Designd primer referred to as “Tag2T790M” in Fig.10
gctggagctctgcagctaagcaggtactgggagccaat

SEQ ID NO: 22 - DNA, 40 bp, Artificial Sequence
  Designd primer referred to as “Tag1L858R” in Fig.10
tcccgcgaaattaatacgacagccaggaacgtactggtga

SEQ ID NO: 23 - DNA, 38 bp, Artificial Sequence; identical to SEQ ID NO: 4
  Designd primer referred to as “Tag2L858R” in Fig.10

SEQ ID NO: 24 - DNA, 18 bp, Artificial Sequence
  Designd primer referred to as “19del_35_49_647” in Fig.10
ggagatgttttgatagcg

SEQ ID NO: 25 - DNA, 18 bp, Artificial Sequence
  Designd primer referred to as “19del_36_50_647” in Fig.10
cggagatgtcttgatagc

SEQ ID NO: 26 - DNA, 18 bp, Artificial Sequence
  Designd primer referred to as “19del_40_51_647” in Fig.10
tcggagatgattccttga

SEQ ID NO: 27 - DNA, 18 bp, Artificial Sequence
  Designd primer referred to as “19del_40_57_647” in Fig.10
tggctttcgattccttga

SEQ ID NO: 28 - DNA, 19 bp, Artificial Sequence
  Designd primer referred to as “19del_WT_488” in Fig.10
tgttgcttctcttaattcc

SEQ ID NO: 29 - DNA, 17 bp, Artificial Sequence
  Designd primer referred to as “T790M_Mut_LNA_647” in Fig.10
atgagctgcatgatgag

SEQ ID NO: 30 - DNA, 16 bp, Artificial Sequence
  Designd primer referred to as “T790M_WT_LNA_488” in Fig.10
tgagctgcgtgatgag

SEQ ID NO: 31 - DNA, 18 bp, Artificial Sequence
  Designd primer referred to as “L858R_Mut_LNA_647” in Fig.10
gtttggcccgcccaaaat

SEQ ID NO: 32 - DNA, 18 bp, Artificial Sequence
  Designd primer referred to as “L858R_WT_LNA_488” in Fig.10
gtttggccagcccaaaat

SEQ ID NO: 33 - DNA, 17 bp, Artificial Sequence
  Designd primer referred to as “T2582A_Mut_LNA_647” in Fig.10
cacccagctgtttggcc

SEQ ID NO: 34 - DNA, 17 bp, Artificial Sequence
  Designd primer referred to as “T2582A_WT_LNA_488” in Fig.10
cacccagcagtttggcc

SEQ ID NO: 35 - DNA, 21 bp, Artificial Sequence
  Designd primer referred to as “19del_BEAM_PE_b” in Fig.10
agcaaagcagaaactcacatc

SEQ ID NO: 36 - DNA, 17 bp, Artificial Sequence
  Designd primer referred to as “T790M_BEAM_PE_b” in Fig.10
cggacatagtccaggag

SEQ ID NO: 37 - DNA, 21 bp, Artificial Sequence
  Designd primer referred to as “L858R_BEAM_PE_b” in Fig.10
atgcctccttctgcatggtat

SEQ ID NO: 38 - DNA, 58 bp, Artificial Sequence
  Designd primer referred to as “e19ion_A_F5_01” in Fig.12
ccatctcatccctgcgtgtctccgactcagacagtaagttaaaattcccgtcgctatc

SEQ ID NO: 39 - DNA, 58 bp, Artificial Sequence
  Designd primer referred to as “e19ion_A_F5_05” in Fig.12
ccatctcatccctgcgtgtctccgactcagagcataagttaaaattcccgtcgctatc

SEQ ID NO: 40 - DNA, 58 bp, Artificial Sequence
  Designd primer referred to as “e19ion_A_F5_09” in Fig.12
ccatctcatccctgcgtgtctccgactcagcacgtaagttaaaattcccgtcgctatc

SEQ ID NO: 41 - DNA, 58 bp, Artificial Sequence
  Designd primer referred to as “e19ion_A_F5_13” in Fig.12
ccatctcatccctgcgtgtctccgactcagcgctcaagttaaaattcccgtcgctatc

SEQ ID NO: 42 - DNA, 58 bp, Artificial Sequence
  Designd primer referred to as “e19ion_A_F5_20” in Fig.12
ccatctcatccctgcgtgtctccgactcagtatgcaagttaaaattcccgtcgctatc

SEQ ID NO: 43 - DNA, 58 bp, Artificial Sequence
  Designd primer referred to as “e19ion_A_F5_24” in Fig.12
ccatctcatccctgcgtgtctccgactcagtgactaagttaaaattcccgtcgctatc

SEQ ID NO: 44 - DNA, 41 bp, Artificial Sequence
  Designd primer referred to as “e19ion_trP1_R” in Fig.12
cctctctatgggcagtcggtgatgacccccacacagcaaag

SEQ ID NO: 45 - DNA, 54 bp, Artificial Sequence
  Designd primer referred to as “e20ion_A_F5_01” in Fig.12
ccatctcatccctgcgtgtctccgactcagacagtgcatctgcctcacctccac

SEQ ID NO: 46 - DNA, 54 bp, Artificial Sequence
  Designd primer referred to as “e20ion_A_F5_05” in Fig.12
ccatctcatccctgcgtgtctccgactcagagcatgcatctgcctcacctccac

SEQ ID NO: 47 - DNA, 54 bp, Artificial Sequence
  Designd primer referred to as “e20ion_A_F5_09” in Fig.12
ccatctcatccctgcgtgtctccgactcagcacgtgcatctgcctcacctccac

SEQ ID NO: 48 - DNA, 54 bp, Artificial Sequence
  Designd primer referred to as “e20ion_A_F5_13” in Fig.12
ccatctcatccctgcgtgtctccgactcagcgctcgcatctgcctcacctccac

SEQ ID NO: 49 - DNA, 54 bp, Artificial Sequence
  Designd primer referred to as “e20ion_A_F5_20” in Fig.12
ccatctcatccctgcgtgtctccgactcagtatgcgcatctgcctcacctccac

SEQ ID NO: 50 - DNA, 54 bp, Artificial Sequence
  Designd primer referred to as “e20ion_A_F5_24” in Fig.12
ccatctcatccctgcgtgtctccgactcagtgactgcatctgcctcacctccac

SEQ ID NO: 51 - DNA, 43 bp, Artificial Sequence
  Designd primer referred to as “e20ion_trP1_R” in Fig.12
cctctctatgggcagtcggtgatagcaggtactgggagccaat

SEQ ID NO: 52 - DNA, 55 bp, Artificial Sequence
  Designd primer referred to as “e21ion_A_F15_01” in Fig.12
ccatctcatccctgcgtgtctccgactcagacagtagccaggaacgtactggtga

SEQ ID NO: 53 - DNA, 55 bp, Artificial Sequence
  Designd primer referred to as “e21ion_A_F15_05” in Fig.12
ccatctcatccctgcgtgtctccgactcagagcatagccaggaacgtactggtga

SEQ ID NO: 54 - DNA, 55 bp, Artificial Sequence
  Designd primer referred to as “e21ion_A_F15_09” in Fig.12
ccatctcatccctgcgtgtctccgactcagcacgtagccaggaacgtactggtga

SEQ ID NO: 55 - DNA, 55 bp, Artificial Sequence
  Designd primer referred to as “e21ion_A_F15_13” in Fig.12
ccatctcatccctgcgtgtctccgactcagcgctcagccaggaacgtactggtga

SEQ ID NO: 56 - DNA, 55 bp, Artificial Sequence
  Designd primer referred to as “e21ion_A_F15_20” in Fig.12
ccatctcatccctgcgtgtctccgactcagtatgcagccaggaacgtactggtga

SEQ ID NO: 57 - DNA, 55 bp, Artificial Sequence
  Designd primer referred to as “e21ion_A_F15_24” in Fig.12
ccatctcatccctgcgtgtctccgactcagtgactagccaggaacgtactggtga

SEQ ID NO: 58 - DNA, 43 bp, Artificial Sequence
  Designd primer referred to as “e21ion_trP1_R” in Fig.12
cctctctatgggcagtcggtgattgcctccttctgcatggtat

SEQ ID NO: 59 - DNA, 56 bp, Artificial Sequence
  Designd primer referred to as “Kras_e2_A_F15_01” in Fig.12
ccatctcatccctgcgtgtctccgactcagacagtaggcctgctgaaaatgactga

SEQ ID NO: 60 - DNA, 56 bp, Artificial Sequence
  Designd primer referred to as “Kras_e2_A_F15_05” in Fig.12
ccatctcatccctgcgtgtctccgactcagagcataggcctgctgaaaatgactga

SEQ ID NO: 61 - DNA, 56 bp, Artificial Sequence
  Designd primer referred to as “Kras_e2_A_F15_09” in Fig.12
ccatctcatccctgcgtgtctccgactcagcacgtaggcctgctgaaaatgactga

SEQ ID NO: 62 - DNA, 56 bp, Artificial Sequence
  Designd primer referred to as “Kras_e2_A_F15_13” in Fig.12
ccatctcatccctgcgtgtctccgactcagcgctcaggcctgctgaaaatgactga

SEQ ID NO: 63 - DNA, 56 bp, Artificial Sequence
  Designd primer referred to as “Kras_e2_A_F15_20” in Fig.12
ccatctcatccctgcgtgtctccgactcagtatgcaggcctgctgaaaatgactga

SEQ ID NO: 64 - DNA, 56 bp, Artificial Sequence
  Designd primer referred to as “Kras_e2_A_F15_24” in Fig.12
ccatctcatccctgcgtgtctccgactcagtgactaggcctgctgaaaatgactga

SEQ ID NO: 65 - DNA, 46 bp, Artificial Sequence
  Designd primer referred to as “Kras_e2_trP1_R” in Fig.12
cctctctatgggcagtcggtgatattgttggatcatattcgtccac

(19) United States
(12) Patent Application Publication
(10) Pub. No.: US 2014/0227706 A1
(43) Pub. Date: Aug. 14, 2014

(54) Method for assessing progression of clinical state of malignant neoplasm by quantitative detection of DNA in blood

(71) Applicants: Kikuya Kato, Osaka-shi (JP); Fumio Imamura, Osaka-shi (JP); Kazuya Taniguchi, Osaka-shi (JP); Toru Kumagai, Osaka-shi (JP); Junji Uchida, Osaka-shi (JP); Kazumi Nishino, Osaka-shi (JP)

(72) Inventors: Kikuya Kato, Osaka-shi (JP); Fumio Imamura, Osaka-shi (JP); Kazuya Taniguchi, Osaka-shi (JP); Toru Kumagai, Osaka-shi (JP); Junji Uchida, Osaka-shi (JP); Kazumi Nishino, Osaka-shi (JP)

(73) Assignees: DNA CHIP RESEARCH INC., Yokohama-shi, Kanagawa (JP); Osaka Prefectural Hospital Organization, Osaka-shi, Osaka (JP)

(21) Appl. No.: 14/118,098

(22) PCT Filed: May 16, 2012
(86) PCT No.: PCT/JP2012/062543

§ 371 Date: Apr. 28, 2014

(30) Foreign Application Priority Data
May 16, 2011 (JP) 2011-109498

(51) Int. Cl. C12Q 1/68

(52) U.S. Cl. CPC C12Q 1/6881
USPC 435/6.12

(57) ABSTRACT

The object of the invention is to provide a method for quantitatively assessing the degree of progression of a malignant neoplasm in a patient who has been medicated. Provided is a method for assessing the progression of the clinical state of a malignant neoplasm in a subject who has been administered with a medicine for treating the malignant neoplasm, the method being characterized by comprising: (1) a step of determining the ratio of DNA molecules having an activation mutation that serves as an activation marker for the medicine to DNA molecules having a normal marker gene in DNA in the blood from the subject; (2) a step of determining the ratio of DNA molecules having a resistance mutation that serves as a resistance marker for the medicine to DNA molecules having a normal marker gene in the DNA in the blood from the subject; and (3) a step of comparing a value obtained in the step (2) with a value obtained in the step (1) to thereby assess whether or not the malignant neoplasm in the subject has acquired resistance to the treatment with the medicine.

### CROSS REFERENCE TO RELATED APPLICATIONS

This application is a United States national phase of co-pending international patent application No. PCT/JP2012/062543, filed May 16, 2012, which claims benefit of Japanese Patent Application No. 2011-109498, filed May 16, 2011, the disclosures of which are incorporated herein by reference.

### FIELD OF THE INVENTION

The present invention relates to a method for assessing the progression of the clinical state of a malignant neoplasm in a subject who has been administered with a medicine for treating the malignant neoplasm and a kit used for this assessment method.

### BACKGROUND OF THE INVENTION

Various remedies such as surgical treatment, chemotherapy and radiation therapy have conventionally been performed for diseases caused by malignant neoplasms (malignant tumors) including cancer, brain tumor, and hematopoietic malignancies such as leukemia and lymphoma depending on the site of tumor and the degree of progression thereof.

Among these multiple remedies, the surgical treatment and the radiation therapy are effective in topical treatment while the chemotherapy for cancer, for example, is effective in systemic treatment because an anticancer agent can be administered to the blood intravenously or orally in order to suppress the division of cancer cells that are present in the entire body and destroy them so that therapeutic effects can be expected regardless of where cancer cells are located in the body.

Moreover, since a large number of therapeutic agents have been developed for specific cancer, leukemia, etc., the chemotherapy has been used for treating lesions and advanced cancer that have spread to the entire body by combining it with the surgical treatment and the radiation therapy.

In the chemotherapy, therapeutic agents for specific cancer, leukemia, etc. are very effective at the beginning of the treatment, yet there are some possibility that as the treatment is continued malignant neoplasms become resistant to those agents such that no further therapeutic effect can be expected. For example, it is known that Gefitinib (Irresa), an epidermal growth factor receptor tyrosine kinase inhibitor (EGFR TKI), shows antitumor effects at a high successful rate of 70-80% for some patients with non-small cell lung cancer (Non-Patent Literature 1). However, resistance finally occurs to all the patients administered with Gefitinib; many of those cases appear within about one year of the administration; and tumor enlarges again after the resistance occurred (Non-Patent Literature 2).

### PRIOR ART LITERATURE

### Non-Patent Literature

Non-Patent Literature 1: Riely, G. J., Politi, K. A., and Miller, V. A., et al. (2006). Update on epidermal growth factor receptor mutations in non-small cell lung cancer. Clin. Cancer Res. 12, 7232-7241.
Non-Patent Literature 2: Sequist, L. V., Bell, D. W., Lynch, T. J., and Haber, D. A. (2007). Molecular predictors of response to epidermal growth factor receptor antagonists in non-small-cell lung cancer. J. Clin. Oncol. 25, 587-595.

Such acquisition of resistance to therapeutic agents is confirmed at present by collecting part of a living tissue or organ from a patient and performing a biopsy, yet the biopsy is invasive and puts too heavy a burden on a patient and, therefore, is not preferable.

### SUMMARY OF THE INVENTION

The present invention has been designed in view of the abovementioned circumstance, and the object of the present invention is to provide a method for quantitatively assessing the degree of progression of a malignant neoplasm in a patient who has been medicated by noninvasively observing the presence or absence of resistance to a specific medicine for the malignant neoplasm.

The object of the invention is also to provide a kit available for the abovementioned assessment method.

The present inventors found that a very small amount of a resistance mutation, which caused drug resistance, was present before administering an anticancer agent in some cases of non-small cell lung cancer and believed it possible that a very small amount of a resistance mutation was present in a primary lesion because the frequency of identification of this mutation was high among patients whose clinical stages were advanced. Furthermore, based on the report that the duration of response significantly declined in cases in which resistance mutations were detected from histological examinations conducted immediately before administering medicines, the present inventors paid attention to the possibility that a very small amount of a resistance mutation found in a primary lesion could be used as a resistance predictive factor for medicines.

As a result of intensive studies with intent to solve the abovementioned problems, the present inventors found that it was possible to quantitatively assess the degree of progression of a malignant neoplasm in a patient by detecting a resistance mutation in a primary lesion using BEAMing, a high-sensitivity minute-mutation detecting method, and thereby shedding light on the relationship between this resistance mutation and the duration of response of medicines.

Specifically, according to a first main aspect of the invention, a method for assessing the progression of the clinical state of a malignant neoplasm in a subject who has been administered with a medicine for treating the malignant neoplasm is provided, wherein the method is characterized by comprising: (1) a step of determining the ratio of DNA molecules having an activation mutation that serves as an activation marker for the medicine to DNA molecules having a normal marker gene in DNA in the blood from the subject; (2) a step of determining the ratio of DNA molecules having a resistance mutation that serves as a resistance marker for the medicine to DNA molecules having a normal marker gene in the DNA in the blood from the subject; and (3) a step of comparing a value obtained in the step (2) with a value obtained in the step (1).

The abovementioned constitution enables to assess whether or not a malignant neoplasm in a subject has become resistant to the treatment with an administered medicine simply by comparing the ratio of an activation mutation with the ratio of a resistance mutation, and therefore the progression of the clinical state of the malignant neoplasm in the subject can be assesses simply by collecting blood without putting too heavy a burden on the subject. Furthermore, the abovementioned constitution enables to assess the progression of the clinical state of the malignant neoplasm in the subject noninvasively and simply and thereby use the assessment as means for selecting a remedy suitable for the subject.

Furthermore, the method according to the invention can be adopted as a method for providing objective and simple information necessary for managing the health and disease state of each patient.

Furthermore, in one embodiment of the invention, the abovementioned subject in such a method is a patient with non-small cell lung cancer; the abovementioned medicine is an EGFR inhibitor; and the abovementioned normal marker gene is a normal EGFR gene.

In this case, the abovementioned resistance mutation is preferably T790M in an EGFR gene. Moreover, the abovementioned activation mutation is preferably one or more mutations selected from ΔE746-A750, L858R, G719C, G719S and G719A in an EGFR gene.

In one embodiment of the invention, the abovementioned EGFR inhibitor is preferably Gefitinib or Erlotinib.

Moreover, according to another embodiment of the invention, the abovementioned subject is a patient with chronic myeloid leukemia (CML) and the abovementioned medicine is Imatinib in the abovementioned method according to the first main aspect of the invention.

In this case, the abovementioned resistance mutation is preferably T3151. Moreover, the abovementioned activation mutation is preferably bcr-abl.

Furthermore, according to another embodiment of the invention, the abovementioned subject is a patient with lung cancer or pulmonary adenocarcinoma, and the abovementioned medicine is an ALK inhibitor in the abovementioned method according to the first main aspect of the invention.

In this case, the abovementioned resistance mutation is preferably L1195M or C1156Y. Moreover, the abovementioned activation mutation is preferably EML4-ALK.

In one embodiment of the invention, the abovementioned ALK inhibitor is preferably Crizotinib.

Furthermore, according to another embodiment of the invention, the abovementioned step (1) and step (2) are performed by using emulsion PCR in the abovementioned method according to the first main aspect of the invention.

Furthermore, according to a second main aspect of the invention, provided is a kit used for the abovementioned method according to the first main aspect of the invention, wherein the kit is characterized by comprising a primer set used for detecting the abovementioned activation mutation and a primer set used for detecting the abovementioned resistance mutation.

The characteristics and marked operation and effect of the invention other than those described above become obvious for those skilled in the art by referring to the embodiments and drawings of the invention below.

### BRIEF DESCRIPTION OF THE DRAWINGS

FIG. 1 is a schematic view showing an outline of BEAMing in one embodiment of the invention.

FIG. 2 is a schematic view showing an outline of sequence differentiation by fluorometric analyses in one embodiment of the invention.

FIG. 3 is a graph showing analytical results by flow cytometry in one embodiment of the invention.

FIG. 4 is a graph showing the quantification of beads produced by BEAMing using a one base extension method in one embodiment of the invention.

FIG. 5 is a graph showing the measurement of melting temperature in one embodiment of the invention.

FIG. 6 is a graph showing quantification by BEAMing in one embodiment of the invention.

FIG. 7 is a graph showing fractions of mutated fragments quantified by BEAMing in one embodiment of the invention.

FIG. 8 is a graph showing the results of flow cytometry in one embodiment of the invention.

FIG. 9 is a table showing the assessment of progression of the clinical state of a malignant neoplasm in one embodiment of the invention.

FIG. 10 is a table showing primer sets when BEAMing is used in one embodiment of the invention.

FIG. 11 is a table showing the assessment of progression of the clinical state of a malignant neoplasm in one embodiment of the invention.

FIG. 12 is a table showing primer sets when a next generation sequencer is used in one embodiment of the invention.

### DETAILED DESCRIPTION OF THE INVENTION

A description of examples in one embodiment of the invention is given below with reference to drawings.

As described above, the method for assessing the progression of the clinical state of a malignant neoplasm according to the present embodiment in a subject who has been administered with a medicine for treating the malignant neoplasm is characterized by comprising (1) a step of determining the ratio of DNA molecules having an activation mutation that serves as an activation marker for the medicine to DNA molecules having a normal marker gene in DNA in the blood from the abovementioned subject; (2) a step of determining the ratio of DNA molecules having a resistance mutation that serves as a resistance marker for the medicine to DNA molecules having a normal marker gene in the DNA in the blood from the abovementioned subject; and (3) a step of comparing a value obtained in the abovementioned step (2) with a value obtained in the abovementioned step (1) and thereby assessing whether or not the abovementioned malignant neoplasm in the subject has become resistant to the treatment with the abovementioned medicine.

In one embodiment of the invention, the term “malignant neoplasm” or “malignant tumor” as used herein refers to tumor and the like that can be enlarged in the entire body by infiltrating or metastasizing into other tissues. In general, this term refers to cancer, leukemia and the like but is not particularly limited to those diseases but includes any diseases in which cells abnormally proliferate including intraepithelial neoplasm, epithelial dysplasia, adenoma, sarcoma, malignant lymphoma, osteosarcoma, and myosarcoma.

The assessment of “progression of the clinical state caused by a malignant neoplasm” refers to stage classification based on such standards as the size of cancer, metastasis to peripheral lymph nodes and metastasis to remote organs if the disease is cancer though the standards and levels differ depending on types of diseases. Moreover, the standard for assessing the progression of the clinical state is not limited to one kind for various types of diseases, i.e., in the present invention, the assessment of “progression of the clinical state caused by a malignant neoplasm” may be expressed by one progression standard, or the degree of progression of the clinical state may be assessed by combining a plurality of progression standards. Moreover, in one embodiment of the invention, the assessment of “progression of the clinical state caused by a malignant neoplasm” also includes the propriety of administering a specific medicine and the propriety of carrying out a specific remedy.

Moreover, in one embodiment of the invention, the “DNA in the blood from the subject” refers to DNA circulating in the blood, and the “DNA in the blood” as used herein includes circulating tumor DNA (ctDNA). It is generally known that of all DNA molecules in the blood, only a very small amount of ctDNA is present as compared to normal cellular DNA.

Moreover, in one embodiment of the invention, the “marker gene” refers to a target gene for a molecular target medicine and particularly to a causative gene that is the target of a molecular target medicine when the causative gene of a specific disease or a target gene that acts on the causative gene is known. Moreover, in one embodiment of the invention, when the causative gene of a specific disease is caused by a mutation of one gene, the “normal marker gene” refers to the gene (normal gene) in a normal cell. Moreover, when the causative gene of a specific disease is caused by mutations of one or more gene(s) and thereby one or more kinds of proteins become abnormal (e.g., abnormal protein that is not found in a normal cell is found), the “normal marker gene” refers to one or more gene(s) (a group of normal genes) in a normal cell corresponding to the one or more mutated gene(s).

Moreover, in one embodiment of the invention, the “activation mutation that serves as an activation marker for the medicine” refers to a mutation that is present in a target gene or target protein on which a molecular target medicine acts or an abnormal protein itself that is not found in a normal cell. Moreover, in one embodiment of the invention, the “resistance mutation that serves as a resistance marker for the medicine” refers to a mutation that causes drug resistance in a specific disease.

Furthermore, the method according to the invention enables to assess the degree of progression of the clinical state of a patient by quantitatively analyzing an activation mutation and a resistance mutation to a disease-related gene of the patient for a specific medicine.

(Non-Small Cell Lung Cancer)

A description of non-small cell lung cancer is given below as an example. The non-small cell lung cancer is one type of lung cancer, and it is known that epidermal growth factor receptor (EGFR) is involved therein. EGFR is transmembrane glycoprotein having a molecular weight of about 170,000 and is known to be involved in the growth and existence of cancer, and its expression has been confirmed in various types of cancer including non-small cell lung cancer. EGFR exists as a monomer in its inactivated state, forms a dimer when a growth factor such as EGF binds to a receptor, and phosphorylates a tyrosine residue in the intracellular domain of the receptor by making use of ATP at a tyrosine kinase site in the intracellular domain. As a result, the tyrosine phosphorylation of protein located at the downstream of a signaling pathway is brought about in a chain reaction, and a growth signal reaches the nucleus to advance the cell cycle from G1 phase to S phase, resulting in the growth of cells. Furthermore, the activation of EGFR tyrosine kinase accelerates the production of intracellular growth factors such as TGF-α and bFGF, stimulates the growth of cells through an autocrine or paracrine pathway, and promotes angiogenesis by accelerating the production of a vascular endothelial growth factor (VEGF). It is believed that a series of these changes aggravates cancer and advances the clinical state.

In the method according to the invention, the subject for whom the progression of the clinical state caused by a malignant neoplasm can be assessed includes a patient with non-small cell lung cancer. In this case, the medicine administered for the treatment is an EGFR inhibitor represented by Gefitinib, Erlotinib or the like, yet in the method according to the invention, Gefitinib and Erlotinib are preferable.

Gefinitib has Quinazoline as its basic framework, and the mechanism is such that since it is structurally similar to adenine of ATP, Gefinitib reversibly binds to the ATP binding site of an intracellular tyrosine kinase region of EGFR and thereby inhibiting ATP binding and preventing activation.

It is known that Gefinitib shows anticancer effects in some patients with non-small cell lung cancer and that most of those patients have mutations in a gene corresponding to the EGFR tyrosine kinase region. It is believed that this medicine has its effects because these mutations change the structure in such a manner that Gefinitib can easily bind while keeping EGFR in an activated state constantly (activation mutation). This activation mutation is exemplified by deletion mutations in exon 19 of a normal EGFR gene (e.g., ΔE746-A750, ΔE746-T751, ΔE746-A750 (ins RP), ΔE746-T751 (ins A/I), ΔE746-T751 (ins VA), ΔE746-S752 (ins A/V), ΔL747-E749 (A750P), ΔL747-A750 (ins P), ΔL747-T751, ΔL747-T751 (ins P/S), ΔL747-S752 ( ), ΔL747-752 (E746V), ΔL747-752 (E753S), ΔL747-S752 (ins Q), ΔL747-P753, ΔL747-P753 (ins S), and ΔS752-1759), L858R point mutation, G719C, G719S, G719A, V689M, N700D, E709K/Q, S720P, V765A, T783A, N826S, A839T, K846R, L861Q and G863D and includes any mutation as far as it can keep EGFR in an activated state constantly. In one embodiment of the invention, an EGFR gene without mutations is used as a normal marker gene for non-small cell lung cancer.

It is also known that resistance occurs to Gefinitib in patients administered with this medicine. The resistance occurs within about one year of the administration in most cases, and tumor is enlarged after the acquisition of the resistance. In about half cases, this is caused by the replacement of threonine, which is an amino acid at position 790 of the EGFR gene, with methionine (resistance mutation, T790M point mutation). The crystallography shows the possibility that the abovementioned change in amino acids, i.e., the replacement with methionine having a large molecular weight causes steric hindrance and thereby lowers the affinity of Gefitinib to EGFR tyrosine kinase. In one embodiment of the invention, this resistance mutation is exemplified by D761Y, D770_N771 (ins NPG), D770_N771 (ins SVQ), D770_N771 (ins G), N771T, V769L and S768I in addition to T790M and includes any mutation as far as it causes the acquisition of resistance to Gefitinib.

In one embodiment of the invention, the degree of progression of the clinical state is assessed in patients with non-small cell lung cancer by making use of the relationship between the activation mutation of the EGFR gene and the resistance mutation of the same as described above.

Moreover, in one embodiment of the invention, the ratio of an activation mutation or a resistance mutation to a normal marker gene is determined by a quantitative technique such as the BEAMing method. As used herein, BEAMing refers to a method for performing PCR reaction in oil emulsion to immobilize a PCR product derived from one molecule onto one nano particle, labelling normal and mutated bases at the site with fluorescent dyes and then detecting them. In this BEAMing and a next generation sequencer, DNA is amplified for each molecule by using emulsion PCR, yet in the method according to the invention any quantitative analysis may be used as far as it can quantitatively determine DNA for each molecule. For example, a wide variety of molecular biology techniques can be used including DNA chips, microarray technique, real-time PCR, northern blotting technique, dot blotting technique and quantitative RT-PCR (quantitative reverse transcription-polymerase chain reaction). In BEAMing, each molecule is recovered by using flow cytometry after amplifying DNA for each molecule by emulsion PCR. Moreover, any type of next generation sequencers may be used as far as it can perform DNA synthesis with DNA polymerase using one DNA molecule as a template and detect fluorescence, emitted light or the like for the reaction of each base in order to determine a base sequence real time, and any base recognition method, lead length, reagent, etc. can also be used for a next generation sequencer.

Moreover, in one embodiment of the invention, while the degree of progression of the clinical state can be assessed by using a wide variety of progression standards or determined based on the propriety of administering a specific medicine or the propriety of carrying out a specific remedy as described above, such assessment can also be conducted by, for example, dividing the ratio (value) of a DNA molecule having a resistance mutation to a DNA molecule having a normal marker gene by the ratio (value) of a DNA molecule having an activation mutation to a DNA molecule having a normal marker gene. Besides, any comparison method can be used for such assessment as far as it can reflect the progression of the clinical state.

With regard to the assessment of progression of the clinical state, the number of accumulated samples about the ratio of a DNA molecule having a resistance mutation or an activation mutation to a DNA molecule having a normal marker gene in a patient is preferably large so that the accuracy of the assessment method according to the invention can be increased. Moreover, in the present invention, the configuration may be such that data on such accumulated samples is stored in any database. In other words, the present invention can provide database for storing such data and an analyzer for reading out and executing such data, programs required for comparative analyses and the like. Such an analyzer makes it possible to accumulate data on the ratio of a DNA molecule having a resistance mutation or an activation mutation to a DNA molecule having a normal marker gene for each subject of interest, retrieve and compare accumulated data as needed and thereby assess the progression of the clinical state of the subject with ease at any time.

(Other Diseases)

As described above, in one embodiment of the invention, the degree of progression of the clinical state of a patient with non-small cell lung cancer can be assessed by quantitatively analyzing an activation mutation and a resistance mutation to EGFR of the patient for gefitinib. For the method according to the invention, the disease of interest is not particularly limited to non-small cell lung cancer, i.e., the present invention can be applied to any disease in which a medicine for the disease is known and there are a causative gene (or activation mutation) and a resistance mutation for the disease, such disease including chronic myeloid leukemia/Imatinib and lung cancer (pulmonary adenocarcinoma)/Crizotinib.

For example, chronic myeloid leukemia (CML) is one kind of chronic leukemia that causes the abnormal division of hematopoietic stem cells in the bone marrow, wherein mutual translocation occurs between chromosome 9 (ABL gene) and chromosome 22 (BCR gene) in a hematopoietic stem cell in the bone marrow, and a resultant chimera gene (BCR-ABL gene) produces Bcr-Abl protein, resulting in this disease (activation mutation). Bcr-Abl protein accelerates cell growth signals by its tyrosine kinase activity and thereby causes white blood cells to proliferate in a disordered state.

As a chemotherapy agent for this chronic myeloid leukemia, Imatinib (Gleevec) that acts on Bcr-Abl protein has been used for many patients, and it is known that resistance to Imanitib occurs in some patients. As to those patients, it is known that the shape of Bcr-Abl protein to which Imanitib binds is genetically mutated such that Imanitib can no longer bind thereto (resistance mutation). This resistance mutation includes, but is not limited to, T3151, yet any mutation that causes the acquisition of resistance to Imatinib can also be included in the resistance mutation.

Moreover, lung cancer, particularly lung adenocarcinoma is cancer that occurs in pulmonary gland cells (e.g., ciliated columnar epithelium in the bronchus, alveolus epithelium and exocrine in the bronchus), and in some patients with lung cancer (pulmonary adenocarcinoma), this disease is induced by abnormal protein as a result of a fusion between an EML4 gene and an ALK gene. More specifically, this is caused by the inversion of regions sandwiching the EML4 gene and the ALK gene in human chromosome 2 in such a way as to form a gene in which the 5′ side of the EML4 gene is fused to a portion of the ALK gene that encodes an enzyme activating region. It is known that the resulting EML4-ALK fusion tyrosine kinase is constantly dimerized by using a dimerization region within EML4, and thereby kinase activity is increased so much so that cancer is induced (activation mutation).

As a chemotherapy agent for such patients with lung cancer (pulmonary adenocarcinoma), Crizotinib, an ALK-specific inhibitor, has been used, and it is known that resistance to Crizonitib occurs in some patients. In many cases, it is known that the shape of EML4-ALK protein on which Crizotinib acts is genetically changed so that this medicine can no longer act on this protein (resistance mutation). This resistance mutation includes, but is not limited to, L1195M and C1156Y, and any mutation that causes the acquisition of resistance to Crizotinib can also be included in the resistance mutation.

(Kit)

In one embodiment of the invention, provided is a kit used in a method for assessing the progression of the clinical state caused by a malignant neoplasm in a subject who has been administered with a medicine for treating the malignant neoplasm, wherein the kit is characterized by comprising a primer set used for detecting the abovementioned activation mutation and a primer set used for detecting the abovementioned resistance mutation.

In one embodiment of the invention, the primer set used for detecting the abovementioned activation mutation or resistance mutation is not particularly limited as far as it is a forward primary set or reverse primer set capable of amplifying a DNA molecule or gene of interest by means of any PCR. Moreover, the primer used in the kit according to the present invention is not particularly limited as far as it is capable of specifically detecting a gene of interest, but preferably it is oligonucleotide having 12-26 bases. Its base sequence is determined based on sequence information about each human gene. A primer having a determined sequence can be produced by a DNA synthesizer. In one embodiment of the invention, emulsion PCR is used as described below, yet PCR is not particularly limited to this method. Moreover, the length and sequence of a primer can appropriately be designed by those skilled in the art. Furthermore, in one embodiment of the invention, the “primer set” may include a probe used for detecting a mutated site as needed depending on PCR to be selected.

### EXAMPLES

A description of the present invention is given below in more detail with reference to examples, yet the present invention is not particularly limited to those examples.

### Experimental Approach and Materials

The following is a description of an experimental approach and materials used for the invention. Although the following experimental approach is used in the present embodiment, similar results can be achieved by other experimental approaches as well.

1. Isolation and Quantification of Genomic DNA

1-1. Extraction and Purification of DNA from the Blood Plasma of Healthy Individuals and Patients with Lung Cancer

We first put 4-5 mL of blood (two centrifuge tubes with EDTA) in a 15 mL centrifuge tube and centrifuged with a large centrifugal machine (LX-120) at 800G for 10 minutes. Since the blood was separated into three layers (blood plasma, white blood cells and red blood cells) by the centrifugation, we collected the blood plasma in a 2 mL microcentrifuge tube while paying attention not to sucking up white blood cells. Next, we centrifuged at 16000G for 10 minutes in order to remove remaining cell components. We then collected the supernatant after the centrifugation as a blood plasma sample (1.2 mL). Subsequently, we purified 400 uL of the blood plasma with Agencourt Genfind V2. We first added 18 μL of proteinase K and 400 μL of the blood plasma to 800 μL of Lysis buffer solution and then mixed them. We then incubated this mixture at 37° C. for 10 minutes. Next, we added 600 μL of binding buffer, throughly performed pipetting and then incubated at room temperature for 5 minutes. We set this solution on a magnet and washed with wash buffer 1 twice and then with wash buffer 2 twice. We throughly removed wash buffer and then added elution buffer to elute DNA.

1-2. Extraction and Purification of DNA from Lung Tumor Samples

We used lung cancer tissue samples presented by Research Institute, Osaka Medical Center for Cancer and Cardiovascular Diseases. Based on the previous studies, we had confirmed typical mutations of EGFR (deletion mutation, point mutation (L858R, G719A, L861Q, T790M)) for these cases by using direct sequencing or SNaPshot reaction. We extracted genomic DNA from these lung cancer samples. We first crushed a frozen sample in a solution of 1 mL of lysis buffer (50 mM NaCl, 20 mM EDTA, 10 mM Tris-HCl pH7.5, 0.2% SDS) and 10 μL of proteinase K (20 mg/mL, Roche) with Mixer Mill MM 300 (QIAGEN), further added 2 mL of lysis buffer and 20 μL of proteinas K and then stirred at 55° C. for 3 hours. Next, we added 3 mL of Phenol:Chloroform:Isoamyl Alcohol 25:24:1 (nacalai tesque), stirred at room temperature for 1 hour and then centrifuged at 2,500 rpm for 20 minutes. We collected the upper layer after the centrifugation, added 7.5 mL of 99.5% ethanol (Wako) and 5 μL of 10 mg/mL glycogen (Invitrogen), mixed, allowed it to stand at −20° C. overnight and then centrifuged at 3,500 rpm for 30 minutes. We removed the supernatant after the centrifugation, eluted a pellet with 300 μL of D.W., added an equal amount of Phenol:Chloroform:Isoamyl Alcohol 25:24:1 (nacalai tesque), mixed and then centrifuged at 13,000 rpm for 10 minutes. We collected the upper layer after the centrifugation, added an equal amount of chloroform, mixed and then centrifuged at 13,000 rpm for 10 minutes. We collected the upper layer after the centrifugation, added 2.5 equivalents of 99.5% ethanol (Wako) and 1/10 equivalents of 3M NaOAc, mixed and then centrifuged at 15,000 rpm for 30 minutes. We removed the supernatant after the centrifugation, washed a pellet with 75% ethanol, removed the supernatant and then dried at room temperature for 10 minutes. We added 10-50 μL of D.W. to a pellet thus obtained and then measured the concentration of DNA based on O.D. (260 nm).

2. Preparation of Samples for Quantitative Analysis

In order to confirm the quantitativity of BEAMing, we used a lung cancer sample, which was a T790M mutant type, to prepare templates containing the mutant type at various ratios. We first extracted totalRNA from a frozen sample by the AGPC method (32) using TRIZOL (Invitrogen). We synthesized double-stranded cDNA from this RNA using Superscript III Reverse Transcriptase (Invitrogen) and E. coli DNA polymerase (Invitrogen) and then performed cloning using a Zero Blunt PCR Cloning Kit (Invitrogen). Subsequently, we confirmed each sequence with a sequencer to prepare 100% wild-type template DNA and 100% mutant-type template DNA. We performed PCR using these DNAs as templates, adjusted the concentration and then mixed samples at various ratios to prepare template DNAs containing 100%, 10%, 1%, 0.1%, 0.01% or 0% of the mutant-type.

3. Analysis of Mutation by BEAMing

### 3-1. Amplification of Target Regions

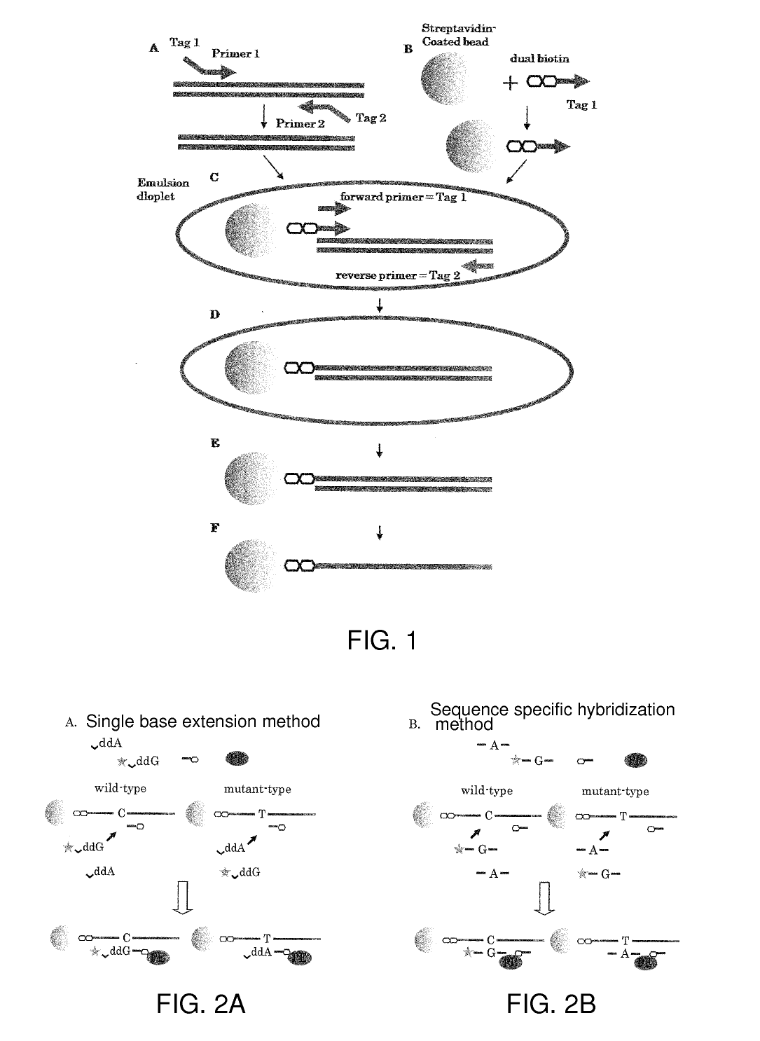

We amplified about 100 bp containing the allele of interest from genomic DNA by PCR (FIG. 1A). We designed primers used for PCR by adding a tag commonly used for BEAMing (Tag 1,5′-tcccgcgaaattaatacgac-3′ (SEQ ID NO: 1), Tag 2,5′-gctggagctctgcagcta-3′ (SEQ ID NO: 2)) on its 5′ side in addition to a genetically specific sequence (primer 1; 5′-tcccgcgaaattaatacgacgcatctgcctcacctccac-3′ (SEQ ID NO: 3), primer 2; 5′-gctggagctctgcagctatgcctccttctgcatggtat-3′ (SEQ ID NO: 4)). To 1.5 μg of the extracted genomic DNA (300 μL of the blood plasma), we added 5 μL of 10×PCR buffer for KOD-plus-(TOYOBO), 5 μL of 2 mM dNTP (TOYOBO), 2 μL of 25 mM MgSO4 (TOYOBO), 1 μL of KOD-plus-(TOYOBO) and 2 μL of 10 pmol/μL primer 1, 2 mix and then adjusted with D.W. such that the total amout became 50 μL. After denaturing it at 94° C. for 2 minutes using a Gene Amp PCR System 9700 Thermal Cycler (Applied Biosystems), we performed PCR for 30 cycles (at 94° C. for 15 seconds, at 62° C. for 10 seconds and at 68° C. for 15 seconds) and then at 72° C. for 5 minutes. We purified PCR products thus obtained using a MinElute PCR Purification Kit (QUIAGEN) and then measured the concentration based on O.D. (260 nm).

### 3-2. Binding of Primers to Magnetic Beads

We let a dual biotinylated probe, which would become a primer at the time of emulsion PCR, bind to a bead coated with streptavidin (FIG. 1B). This oligonucleotide was modified with biotin doubly at the 5′ end by sandwiching a PEG (polyethylene glycol) 18 spacer and a thymidine base (Integrated DNA Technologies). The affinity between biotin and avidin is very strong such that an irreversible bond is formed, and therefore a large number of primers can be bound to the surface of a bead.

We first placed 100 μL of MyOne streptavidin-coated magnetic beads (10 mg/mL; 7-12×109 beads/mL, Invitrogen) in a 1.5 mL microcentrifuge tube, immobilized it on a magnet (DynaMag, Invitrogen) and then removed the supernatant. Subsequently, we added 100 μL of TK buffer (20 mM Tris-HCl (pH8.4), 50 mM KCl), performed mild tapping, immobilized on a magnet again, removed the supernatant and then washed the magnetic beads. After repeating this operation one more time, we suspended the beads in 100 μL of binding buffer (5 mM Tris-HCl (pH7.5), 0.5 mM EDTA, 1M NaCl), added 10 μL of a 100 pmol/μL dual biotinylated probe (5′-tcccgcgaaattaatacgac-3′) (SEQ ID NO: 5), stirred with a vortex mixture and then allowed it to stand at room temperature for 30 minutes. In the meantime, we stirred with the vortex mixture every 10 minutes. Next, we added 10 μL of the 100 pmol/μL dual biotinylated probe again, stirred, allowed it to stand for 10 minutes, washed with 100 μL of IK buffer three times and then suspended in 100 μL of TK buffer.

### 3-3. Preparation of Emulsifier-Oil

We stirred 420 μL of WE09 (Degussa), 200 μL of mineral oil (SIGMA-ALDRICH) 1, and 380 μL of DEC (Degussa) 4 with a vortex mixture and allowed it to stand at room temperaure for 30 minutes.

### 3-4. Emulsion PCR

We adjusted a solution emulsified with the emulsifier-oil and PCR liquid such that a water droplet of the solution contains one of the PCR amplified products prepared in 3-1 and one of the beads prepared in 3-2, and then performed PCR using a DNA tag bound to the bead as a primer (FIGS. 1C and D).

We mixed 1.5 μL of purified template DNA (10 pg/μL), 93.25 μL of D. W., 15 μL of 10×KOD buffer, 15 μL of 2 mM dNTP, 6 μL of 25 mM MgSO4, 9 μL of KOD-plus-, 0.25 μL of 10 pmol/μL forward primer (5′-tcccgcgaaattaatacgac-3′) (SEQ ID NO: 6) and 4 μL of 100 pmol/μL reverse primer (5′-gctggagctctgcagcta-3′) (SEQ ID NO: 7) and then added 6 μL of adjusted magnetic beads after thoroughly stirring them to prepare a PCR liquid having the total amount of 150 μL. Next, we added a 5 mm zirconia bead, 600 μL of the emulsifier-oil and 150 μL of the emulsion PCR liquid to a 2 mL microcentrifuge tube and stirred with Mixer Mill MM 300 (QIAGEN) under the conditions of 15 Hz and 17s. We infused the adjusted liquid into 96-well PCR plates for 50 μL each and then performed PCR reaction under the conditions shown in Table 1.

TABLE 1
Conditions of emulsion PCR

| | | | | | | |
|---|---|---|---|---|---|---|
| 94° C. | 2 min | | | | | |
| ↓ | | | | | | |
| 98° C. | 15 sec | 64° C. | 45 sec | 72° C. | 75 sec | ) 3 cycles |
| ↓ | | | | | | |
| 98° C. | 15 sec | 61° C. | 45 sec | 72° C. | 75 sec | ) 3 Cycles |
| ↓ | | | | | | |
| 98° C. | 15 sec | 58° C. | 45 sec | 72° C. | 75 sec | ) 3 cycles |
| ↓ | | | | | | |
| 98° C. | 15 sec | 57° C. | 45 sec | 72° C. | 75 sec | ) 50 cycles |
| ↓ | | | | | | |
| 10° C. | ∞ | | | | | |

We recovered the PCR reaction liquid to a 2 mL microcentrifuge tube, centrifuged at 15,000 g for 5 minutes and then removed the upper layer. Subsequently, we added 300 μL of breaking buffer (5 mM Tris-HCl (pH7.5), 1% Triton-X 100, 1% SDS, 100 mM NaCl, 1 mM EDTA) and 300 μL of binding buffer (10 mM Tris-HCl (pH7.5), 0.5 mM EDTA, 1M NaCl), stirred with a vortex mixture to break up emulsion (FIG. 1E) and then centrifuged again at 15,000 g for 5 minutes. After removing the upper layer of this solution by using a magnet, we suspended the precipitate in 100 μL of TK buffer and then transferred it to a 1.5 mL tube. Next, after removing the upper layer with a magnet again, we added 500 μL of 0.1M NaOH, stirred with a vortex mixture, allowed it to stand at room temperature for 2 minutes in order to change double-stranded DNA on the bead into a single strand (FIG. 1F) and then removed the upper layer by using a magnet to remove DNA strands that had not been bound to the bead. Finally, we washed it with 100 μL of TK buffer twice and then suspended in 30 μL of D. W.

### 3-5. Sequence Differentiation by Fluorometric Analysis

For the DNA strand bound to the bead, we examined two types of allele specific fluorescence labeling methods in accordance with literatures.

3-5-1. One Base Extension Method

We labeled the DNA allele bound to the bead with different types of fluorescence by single base extension (SBE). The SBE method is a method of hybridizing a probe complimentary to the sequence of an allele of interest up to one base upstream and then incorporating fluorescence-labeled ddNTP by the enzyme reaction of DNA polymerase (FIG. 2A). This method uses enzyme reaction having high allele specificity and, therefore, is excellent at the ability of differentiating alleles and has been widely used as an SNP genotyping technique or the like.

To 14 μL of the bead prepared in 3-4, we first added 2 μL of 10×PCR buffer (Applied Biosystems), 1 μL of Ampli Taq (Applied Biosystems), 0.5 μL of 0.1 mM Cye 5-labeled ddGTP (perkinelmer), 0.5 μL of 0.1 mM FITC-labeled ddATP (perkinelmer) and 2 μL of 10 pmol/μL SBE primer (5′-agccgaagggcatgagctgc-3′) (SEQ ID NO: 8) and then adjusted the total amount to 20 μL. We modified the 5′ end of the SBE primer with biotin (Gene Design Inc.). We had this reacted at 94° C. for 2 minutes, 60° C. for 1 minute and 70° C. for 10 minutes using a Gene Amp PCR System 9700 Thermal Cycler, washed with 100 μL of TK buffer, added 20 μL of binding buffer and 1 μL of 1 mg/mL streptavidin-conjugated phycoerythrin (PE, Invitrogen), performed mild tapping and then allowed it to stand at room temperature for 10 minutes. We washed this bead with 100 μL of TK buffer one time and suspended in 100 μL of TK buffer.

3-5-2. Allele Specific Hybridization

We labeled the DNA allele bound to the bead with different types of fluorescence by allele specific hybridization (ASH). The ASH method is a method of preparing a probe labeled with different types of fluorescence at its 5′ end for oligonucleotide having the allele at its center and then allowing the probe to bind to the target region allele-specifically by hybridization (FIG. 2B). In this study, we modified a probe complimentary to the sequence of mutant-type DNA (mutant-type ASH probe; 5′-atgagctgcatgatg-ag-3′) (SEQ ID NO: 9) with fluorescence Alexa 647 at its 5′ end and also modified a probe complimentary to the sequence of wild-type DNA (wild-type ASH probe; 5′-tgagctgcgtgatgag-3′) (SEQ ID NO: 10) with fluorescence Alexa 488 at its 5′ end (Gene Design Inc.). The number of bases in those probes was respectively set to 17 bp and 16 bp based on the melting temperature (Tm values). It is believed that since it is non-enzymatic reaction, the ASH method is characterized by less fluctuation in reaction and a high labeling rate but is also believed that the ability of differentiating alleles is low and a large number of mismatched hybridization occurs as compared to the SBE method. Therefore, we used locked nucleic acid (LNA) at the allele of the probe for the purpose of enhancing the ability of recognizing a complementary strand. In addition to the abovementioned two types of fluorescent probes, we prepared a probe (biotinylated probe; 5′-cggacatagtccaggag-3′) (SEQ ID NO: 11) complimentary to a consensus sequence for the mutant-type and the wild-type and modified its 5′ end with biotin (Gene Design Inc.)

To 30 μL of the bead prepared in 3-4, we first added 64 μL of 1.5× hybridization buffer (4.5M tetramethylammonium chloride, 75 mM Tris-HCl pH7.5, 6 mM EDTA) and 2 μL each of the abovementioned three types of 5 pmol/μL labeled probes (mutant-type ASH probe, wild-type ASH probe and biotinylated probe), stirred by pipetting, infused into an eight-connected tube for 50 μL each, dissociated the probes at 70° C. for 10 seconds using a Gene Amp PCR System 9700 Thermal Cycler, slowly cooled to 35° C. (0.1° C./s), incubated for 2 minutes and then slowly brought the temperature to room temperature (0.1° C./s). After recovering this reaction solution to a 1.5 mL microcentrifuge tube, we removed the supernatant of this reaction solution using a magnet, suspended in 50 μL of 1× hybridization buffer and then incubated at 48° C. for 5 minutes. After bringing the temperature of the reaction solution to room temperature, we removed the supernatant using a magnet, added 20 μL of binding buffer and 2 μL of 1 mg/mL streptavidin-conjugated phycoerythrin (PE, Invitrogen), performed mild tapping and then allowed it to stand at room temperature for 10 minutes. We washed this bead with 100 μL of TK buffer one time and suspended in 100 μL of TK buffer.

### 3-6. Flow Cytometric Analysis

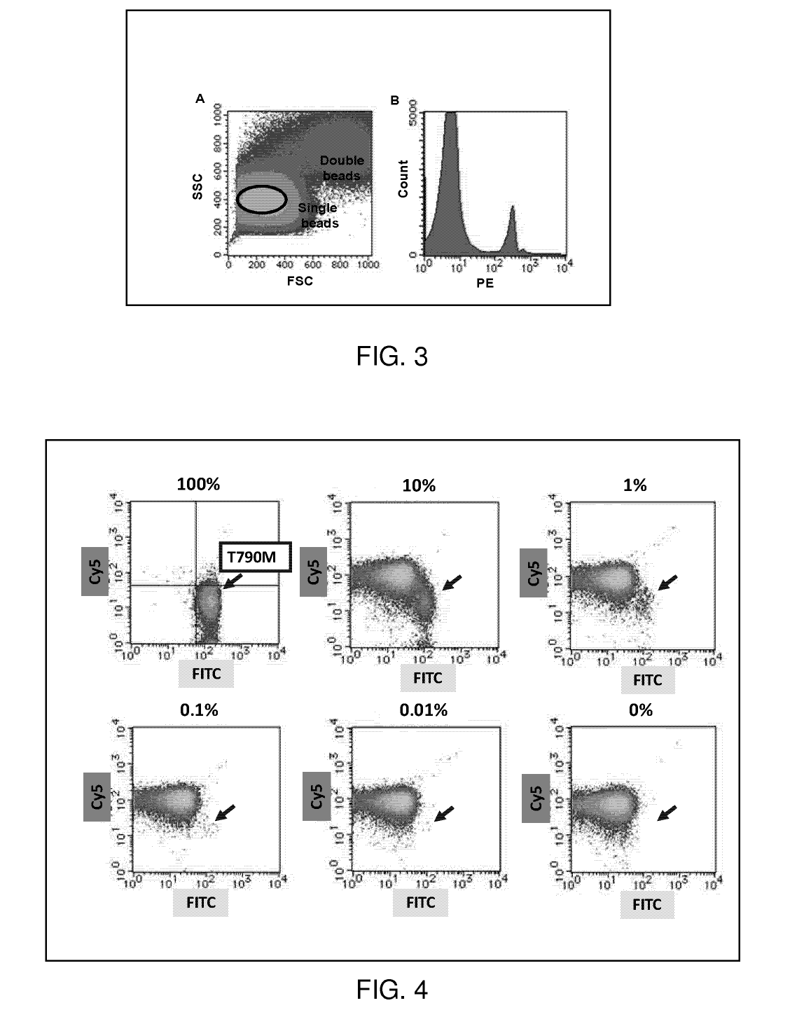

We transferred a beads suspension to a 5 mL polystyrene round-bottom tube (BD Falcon™), diluted it with TK buffer to make a total amount of 1 mL. We analyzed these beads with FACSCalibur (BD Bioscience). We set a flow rate of the beads to about 5,000 events/s and measured individual beads with three colors using a 488 nm argon laser and a 635 nm semiconductor laser. We first selected only single beads based on the values of forward-scatter (FSC) and side-scatter signals (FIG. 3A), and among those beads we used beads on which PE signals were detected (FIG. 3B) for analyses. We calculated the ratio of mutant-type in a sample based on the number of beads on which wild-type signals were detected and the number of beads on which mutant-type signals were detected. Moreover, we used FACS Vantage SE (BD Bioscience) for sorting beads.

While all of the abovementioned experimental protocols relating to “3. Analysis of mutation by BEAMing” describe the detection of T790M mutation in EGFR as an example, these experimental protocols can be appropriately modified for designing primers suitable for the mutation of interest, optimum hybridization conditions and the like within the scope recognizable by those skilled in the art so that any resistance mutation and activation mutation can quantitatively be detected in any disease. For example, in non-small cell lung cancer described in the present specification, resistance mutations such as D761 and N771T in EGFR in addition to T790M and activation mutations such as ΔE746-A750 and L858R in EGFR can quantitatively be detected as well. The present experimental protocols enable to quantitatively detect disease-causing genes (or activation mutations) and resistance mutations for other diseases such as chronic myeloid leukemia and lung cancer (pulmonary adenocarcinoma), yet diseases that can be detected are not particularly limited to the abovementioned diseases.

Determination of Melting Temperature

In order to compare the ability of recognizing a complimentary strand between DNA and LNA, we measured melting temperatures (Tm values) for fullmatch sequence and mismatch sequence. After desalting HPLC-purified oligonucleotides (one end is modified with a fluorescence residue) having mutually complementary sequences, we freeze-dried, dissolved in 1× hybridization buffer such that the amount of each becomes 100 μM, mixed equivalent amounts thereof and then annealed. We confirmed the annealing by nondenaturing polyacrylamide gel electrophoresis and HPLC analysis at a low temperature (20° C.). Subsequently, we raised temperature from 5° C. to 99° C. at a rate of 1° C. per minute and then determined absorbance using UV1650PC/TMSPC-8 (Shimadzu). Table 2 shows the sequences.

TABLE 2
Table 2 Sequence of fluorescent probe

| | sequence | |
|---|---|---|
| LNA fullmatch Alexa 647 | X_N(6)_atgagctgcAtgatgag | (SEQ ID NO: 12) |
| | tactcgacgcactactc | |
| LNA mismatch Alexa 488 | Y_N(6)_tgagctgcGtgatgag | (SEQ ID NO: 13) |
| | tactcgacgcactactc | |
| DNA fullmatch Alexa 647 | X_N(6)_atgagctgcatgatgag | (SEQ ID NO: 14) |
| | tactcgacgcactactc | |
| DNA fullmatch Alexa 488 | Y_N(6)_tgagctgcgtgatgag | (SEQ ID NO: 15) |
| | tactcgacgcactactc | |

Upper case = LNA, Lower case = DNA/X = Alexa647, Y = Alexa488, N(6) = 5′amino C6

Analysis of Sequence

We used Big Dye Terminator ver. 3.1 (Applied Biosystems) for sequence reaction and Agencourt CleanSEQ (BECKMANN COULTER) for the purification. We detected each base sequence using ABI PRISM 3100 Genetic Analyzer (Applied Biosystems) and analyzed it by a BioEdit Sequence Alignment Editor.

(Results)

The following describes the assessment of progression of the clinical state caused by a malignant neoplasm by the method according to the present invention using the abovementioned experimental approach and materials with reference to drawings.

1. Development of Quantitative Analysis for Detecting EGFR T790M Mutation

In order to assess the sensitivity of BEAMing, we performed BEAMing using template DNA having 100%, 10%, 1%, 0.1%, 0.01% and 0% of T790M mutant-type.

1-1. One Base Extension Method

Based on the previous studies, the present inventors have succeeded in BEAMing by the SBE method. FIG. 4 shows the results. In BEMAing, one genome-derived PCR product is bound to one bead. In other words, the ratio of the number of beads separated by fluorescence residues agrees with an allele frequency in each sample.

In this analysis, we measured about 300,000 single beads. Among them, PE signals were detected in about 20,000 beads (PE positive beads), and therefore it is believed that PCR reaction occurred on about 7% of the entire beads. As a result of the fluorescence labeling by the SBE reaction on beads that had PCR reaction, a Cye5 signal was detected for beads containing wild-type DNA sequence and an FITC signal for beads containing mutant-type DNA. In consequence, it was shown that the quantitative measurement of mutant-type DNA is possible up to 0.1% in sensitivity. However, it is difficult to determine mutant-type DNA at a ratio equal to or less than 0.1% by the present method. Since the number of beads used for the quantitative measurement was about 20,000, we cannot expect 1/000 or higher in sensitivity based on the analytical principle. Accordingly, we need to increase the number of beads to be screened significantly in order to increase sensitivity. If the number of beads to be screened is increased, however, the number of beads whose sequence is hard to be differentiated also increases in the background such that it is unlikely to keep accurate quantitativity by the fluorescence intensity according to the SBE method. Therefore, we further improved the fluorescence labeling method for beads with intent to obtain stronger fluorescence signals.

1-2. Allele Specific Hybridization

Based on the previous studies, it has been known that there are a large number of fluorescence residues that are not incorporated onto beads by the SBE method (not shown here). It is believed that one of the causes is that the reaction rate of SBE significantly declines because BEAMing uses reaction with a template immobilized on a solid phase unlike ordinary DNA polymerase extension reaction. On the other hand, the ASH method uses chemical reaction, and therefore it is possible to tip the reaction more toward the possibility that probes can bind by adding an excess amount of probes. However, since the binding force of a probe significantly depends on its sequence and the salt concentration of a solvent at the time of hybridization, the ability of recognizing sequence by a probe needs to be enhanced in order to label fluorescence accurately. In this study, we used LNA at the allele-identification site of a probe. The sugar conformation in nucleosides normally exists in an equilibrium state between N type and S type. The RNA/RNA double strand usually has an A-type helical structure, and its sugar moiety is mainly of N type. On the other hand, the DNA/DNA double strand has a B type helical structure, and its sugar moiety is mainly of S type. Importantly, the sugar conformation should be either N type or S type in order to form a stable double strand. LNA is an artificial nucleic acid analogue in which the ability of forming a double strand is markedly enhanced by methylene cross-linking between an oxygen atom at position 2′ of the sugar moiety and a carbon atom at position 4′ thereof so that the sugar conformation is strictly fixed to N type. However, since a probe used in this study is labeled with a different type of fluorescence residue having a molecular weight of about 600 at its 5′ end, it is possible that the allele-identification ability declines due to tis steric hindrance. Accordingly, we measured melting temperatures (Tm) for fullmatch sequence and mismatch sequence using a fluorescence probe used in this study and its complementary oligonucleotide, and based on the difference we assessed the effect on the allele-identification ability by introducing LNA. The measurement of Tm refers to the measurement of changes in ultraviolet absorption by temperature increase, and the Tm value shows a temperature at which 50% of all the double-stranded DNA molecules are dissociated and is an indicator for the stability of the double strand. Accordingly, by finding the difference (ΔT), we can assess the deviation of the equilibrium at the time of hybridization. As a result of the measurement of Tm values, we confirmed the temperature difference of 1.000° C. between ΔT in LNA (ΔTLNA) and ΔT in DNA (ΔTDNA). Hence, it is clear that a probe introduced with LNA can enhance the allele-recognition ability even when it is modified with a fluorescence residue (FIG. 5).

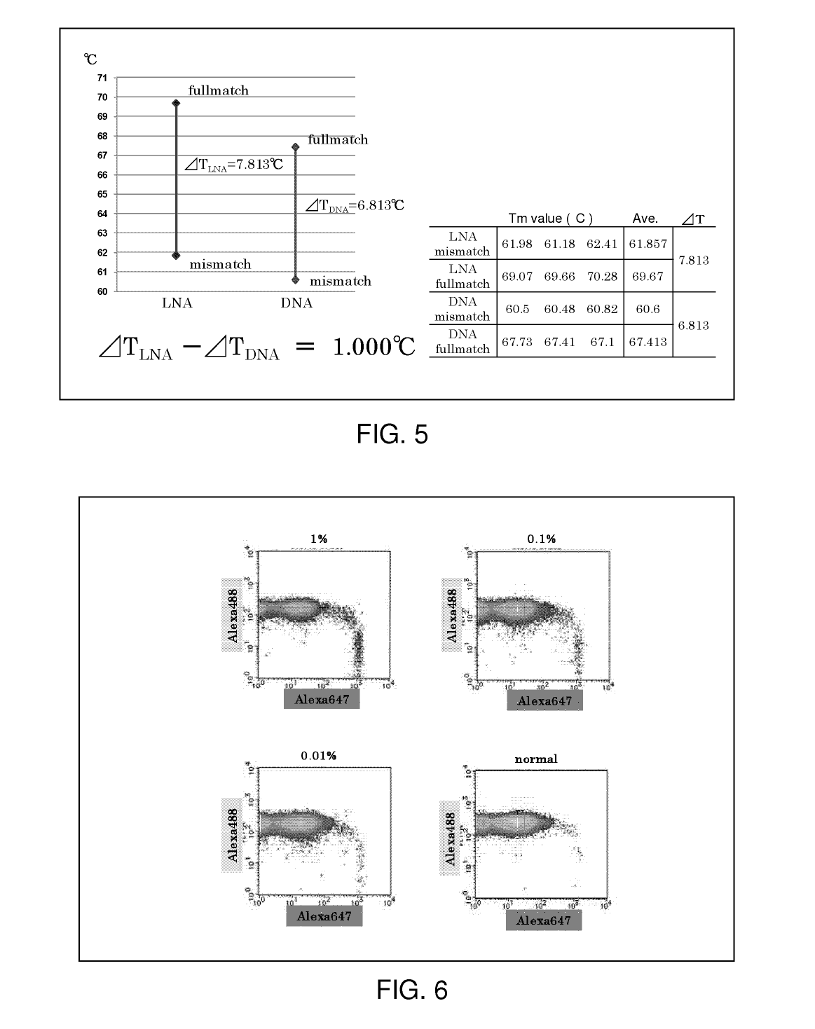

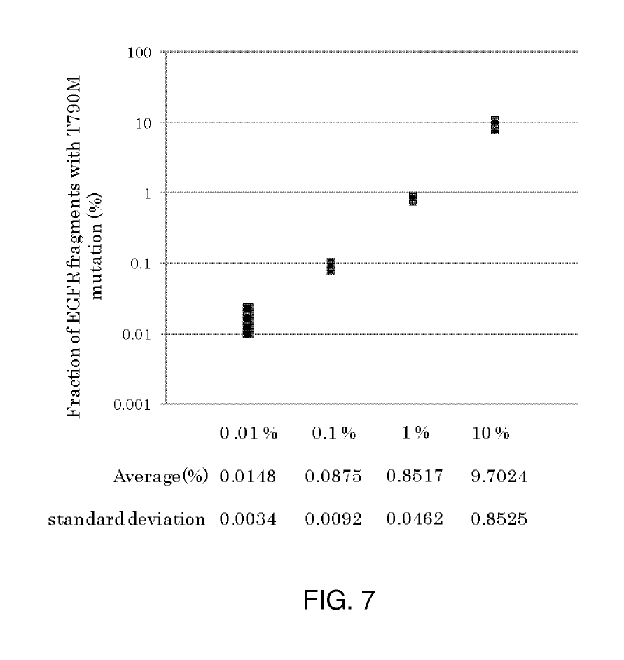

The present experiment has made it clear that the specificity of beads increases at the time of florescence labeling by using LNA. Accordingly, we can expect that by adding fluorescence using a probe whose sequence recognition ability has been enhanced, the fluorescence intensity of individual beads increases, and thereby the separation ability of allele-specific beads can be enhanced. Hence, we performed BEAMing by the ASH method using an LNA probe. FIG. 6 shows the results. In this analysis, we used 500,000 single beads for 1% measurement, 1,000,000 single beads for 0.1% measurement and 5,000,000 single beads each for 0.01% and 0% measurements. In this experiment, PCR reaction occurred on about 7-10% of the entire beads again. As a result of the fluorescence labeling by the ASH reaction on beads that had PCR reaction, an Alexa 488 signal was detected for beads containing wild-type DNA sequence and an Alexa 647 signal for beads containing mutant-type DNA. It is believed that fluorescence probes introduced with LNA were hybridized with a large number of PCR products amplified on beads, and the fluorescence intensity was increased several tens to several hundred times as much compared to the SBE method. Furthermore, we measured samples prepared by mixing at various ratios 10 times each, calculated standard deviation based on the measured values. As a result, it was made clear that measurement was possible while keeping high quantitativity even when a mutation as small as 0.01% had to be detected (FIG. 7). This result suggests that BEAMing makes it possible to accurately recognize a very small number of mutant-type sequences contained in a large number of wild-type sequences without depending on the sequence of a target region. On this occasion, we have succeeded in increasing the number of beads to be screened significantly and thereby enhancing sensitivity by increasing the fluorescence intensity of beads.

Detection of Mutation in Tumor Samples

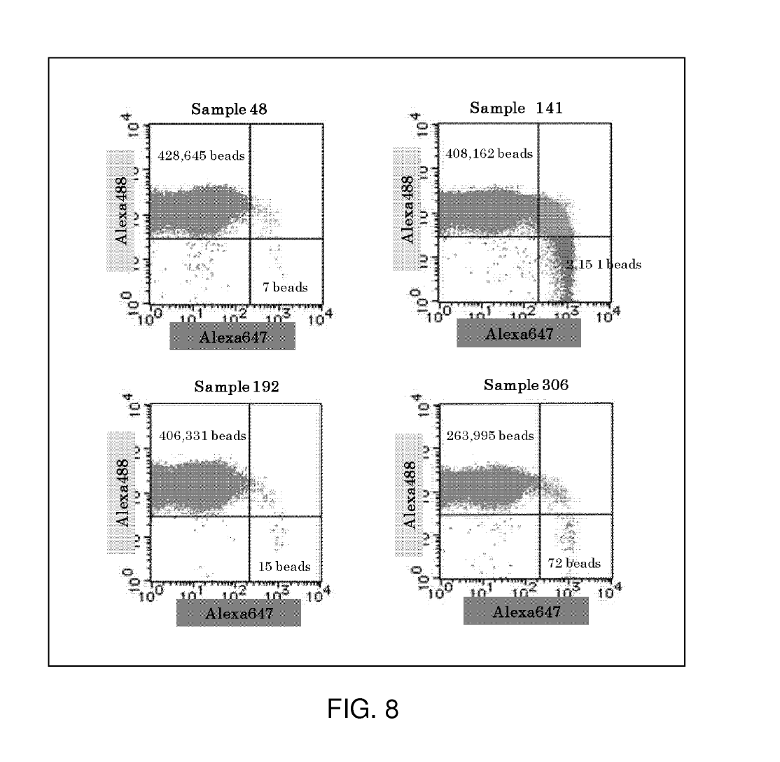

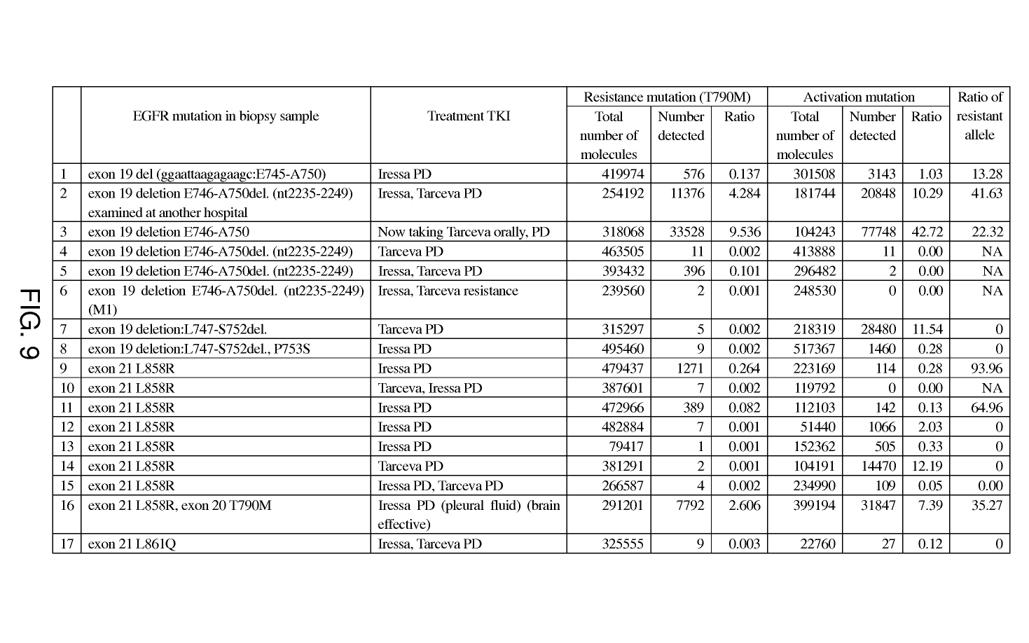

Based on the previous studies, the present inventors have confirmed typical mutations of the EGFR gene (deletion mutation, point mutation (L858R, G719A, L861Q, T790M)) by performing direct sequencing or SNaPshot reaction for 263 cases with primary lung cancer lesions. The T790M mutation is known as Gefitinib resistance mutation and the other mutations as EGFR activation mutation. The SNaPshot reaction is a technique in which a primer complementary up to a region immediately before a mutation-detected site is designed, and after incorporating a fluorescence-labeled nucleotide by extension reaction using DNA polymerase, it is analyzed with a sequencer. This technique enables detection at a higher sensitivity than the direct sequencing and also enables to process a large number of samples. For cases with T790M mutation positive, we detected the T790M mutation using BEAMing. In this analysis, we analyzed about 500,000 PE positive beads. FIG. 8 shows some examples.

Beads in red spots (beads interspersed in the upper left fraction out of four fractions in each graph) show wild-type beads and beads in blue spots (beads interspersed in the lower right fraction out of four fractions in each graph) show mutant-type beads. We calculated the number of beads in each case and indicated positive when the ratio of mutant-type beads is 0.015% or higher. Beads in green spots (beads interspersed in the upper right fraction out of four fractions in each graph) were excluded from the present analysis because it is assumed that two different fragments entered at the same time at the time of emulsion PCR. In FIG. 8, the ratio of mutant-type is respectively 0.0016% and 0.0037% in Sample 48 and Sample 192, and therefore those samples are negative, while it is respectively 0.5270% and 0.0273% in Sample 141 and Sample 306, and therefore those samples are positive.

(Assessment of Progression of Clinical State)

As described above, it has been shown that an activation mutation and a resistance mutation in DNA in the blood plasma can quantitatively be detected. Next, a description of the assessment of progression of the clinical state using the quantitatively obtained activation mutation and resistance mutation is given below with reference to FIGS. 9-11.

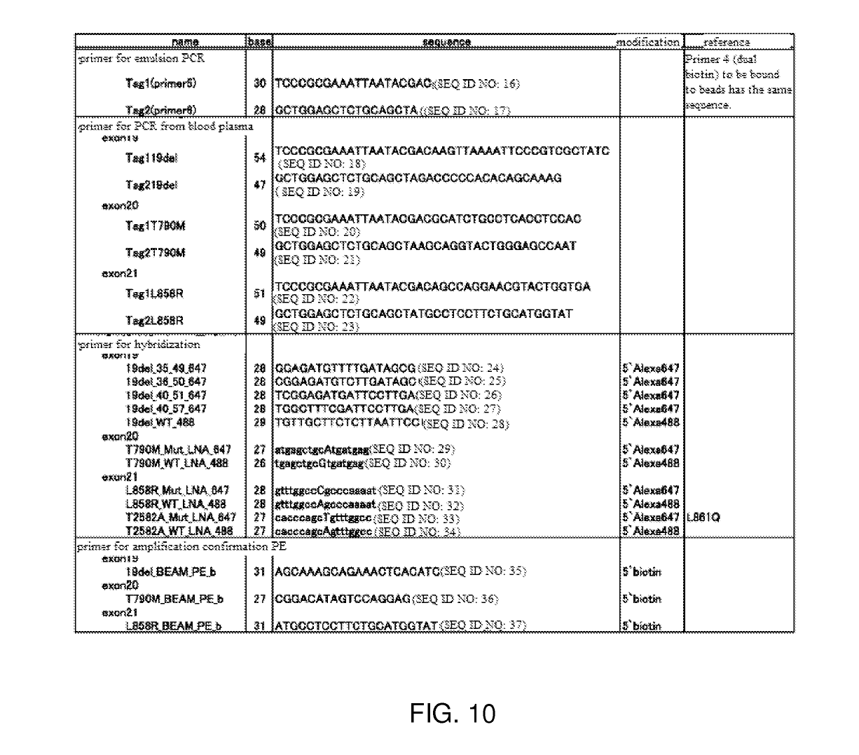

FIG. 9 is a table showing the detection of EGFR mutation in DNA in the blood plasma of some patients with non-small cell lung cancer as an example. In FIG. 9, T790M is used as a resistance mutation, and 19^{th} exon deletion mutation (e.g., ΔE746-A750) and L858R are used as activation mutations. As to the activation mutation, cases in which any of those mutations were detected were combined. FIG. 10 shows various primer sets used for detecting mutations. Sequentially from the top, FIG. 10 shows a primer used for emulsion PCR, a primer used for detecting a mutation of interest in DNA in the blood plasma, a primer for hybridization used for fluorescence labeling and a primer for confirming the amplification of a sequence of interest. It goes without saying that such primer sets can be designed and changed in an appropriate manenr.

Furthermore, in FIG. 9, the progression of the clinical state is assessed by dividing the ratio (value) of a DNA molecule having a resistance mutation to a DNA molecule having a normal marker gene by the ratio (value) of a DNA molecule having an activation mutation to a DNA molecule having a normal marker gene (See the column of the “ratio of resistant allele” in FIG. 9). If a calculable value is found based on the result of this division, the following therapeutic judgment can be made: since a resistance mutation has started being present at a ratio that should be taken into consideration medically, the administration of Gefinitib (Iressa) should be discontinued. Moreover, the numerical value on which the abovementioned judgment is made can be set to 5% or more, 10% or more or the like in an appropriate manner depending on the attributes of a patient such as his/her age and gender, the condition of his/her disease (early, terminal, etc.) and information about the administration of a medicine (type of medicine, administration period, dosage, etc.)

Moreover, in FIG. 9, the ratio of a DNA molecule having a resistance mutation to a DNA molecule having a normal marker gene is calculated based on the ratio of the number of EGFR molecules having the T790M mutation to the total number of EGFR molecules detected by BEAMing (See the column of the “resistance mutation (T790M)” in FIG. 9). For example, as to a patient of Sample No. 1, it is shown that the total number of EGFR molecules is 419974, the number of EGFR molecules having the T790M mutation 576 and the ratio 0.137. Similarly, the ratio of a DNA molecule having an activation mutation to a DNA molecule having a normal marker gene is calculated based on the ratio of the number of EGFR molecules having any one of activation mutations to the total number of EGFR molecules detected by BEAMing (See the column of the “activation mutation” in FIG. 9). For example, as to the patient of Sample No. 1, it is shown that the total number of EGFR molecules is 301508, the number of EGFR molecules having any one of activation mutations 3143 and the ratio 1.03.

Accordingly, the clinical state can be assessed by dividing the ratio (value) thus obtained of a DNA molecule having a resistance mutation to a DNA molecule having a normal marker gene by the ratio (value) of a DNA molecule having an activation mutation to a DNA molecule having a normal marker gene (the result of the division: 13.28 in the patient of Sample No. 1).

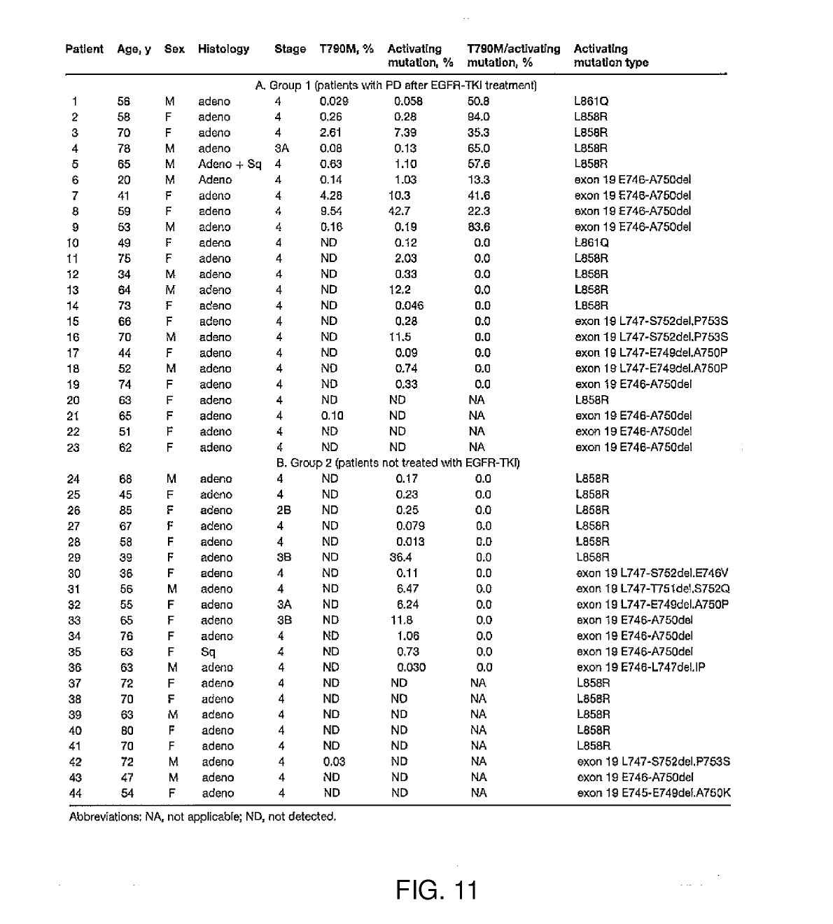

Moreover, FIG. 11 has arranged patient groups including patients in FIG. 9, wherein Group 1 is a group of patients with progressive disease (PD) who were treated with EGFR-TKI, and Group 2 is a group of patients who were not treated with EGFR-TKI. In the drawing, the term “adeno” refers to “adenocarcinoma, the term “Sq” refers to “squamous cell carcinoma,” and the term “adeno+Sq” refers to “adenosquamous carcinoma.” As is obvious from the drawing, the ratio of a resistance mutation (T790M) to activation mutations is 0.0 or NA in all of the patients in Group 2, and therefore the assessment is such that the degree of progression of the clinical state is low, or no resistance mutation is present at a ratio that should be taken into consideration medically. On the other hand, the ratio of a resistance mutation (T790M) to activation mutations is high in patients 1-9 in Group 1, which shows that the clinical state is progressing, and this result is in synch with the fact that they are patients with PD.

(Assessment of Progression of Clinical State when Next Generation Sequencer is Used)

As described above, it has become clear that an activation mutation and a resistance mutation in DNA in the blood plasma can quantitatively be detected by using BEAMing and that the progression of the clinical state of a patient can be assessed by using quantitatively obtained activation mutation and resistance mutation. Next, the following describes the quantitative detection of an activation mutation and a resistance mutation by using a next generation sequencer in place of BEAMing as well as the assessment of progression of the clinical state of a patient based on the quantitative ratio of those mutations.

We amplified each of EGFR exons 19-21 obtained from DNA in the blood plasma of patients with lung cancer by PCR. The reaction liquid used for the amplification reaction is as follows.

| | |
|---|---|
| dH20 | 60 uL |
| 10 x KOD-plus-Buffer | 10 uL |
| 2 mM dNTPs | 10 uL |
| 25 mM MgSO4 | 4 uL |
| PrimerMix (5 uM each) | 4 uL |
| KOD-plus- | 2 uL |
| DNA in blood plasma (equivalent to | 10 uL |
| 400 uL of blood plasma) | |

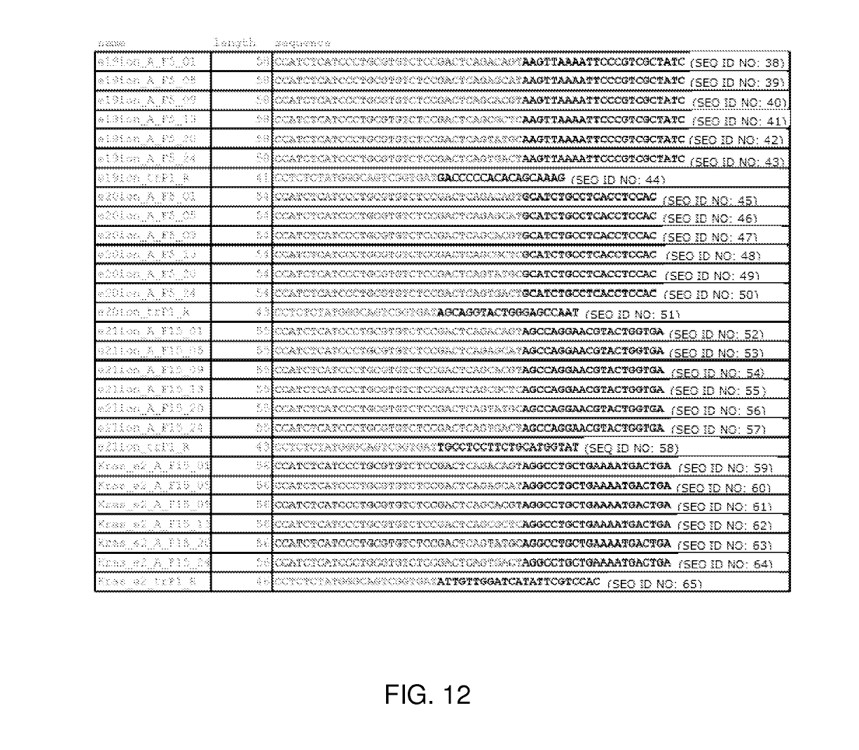

PrimerMix consists of forward and reverse primers for each exon. FIG. 12 shows each sequence of PrimerMix. PCR reaction conditions are as follows.

| | |
|---|---|
| 94dC | 2 minutes |
| 94dC | 15 seconds |
| 62dC | 30 seconds × 40 cycles |
| 68dC | 50 seconds |
| 16dC | Warmth retained |

We purified a sample after the reaction by QIA cube (MinElute PCR purification kit). We then measured the quantity of amplified DNA molecules by NanoDrop, mixed equal amounts of amplified fragments and then prepared a library for a next generation sequencer (Ion Torrent Personal Genome Machine) in accordance with a protocol described in the instrument instructions for use. We performed sequencing reaction for this library using a PGM316 chip. We performed the sequencing reaction for 100,000 molecules or more for each exon molecule to look for mutations. Table 3 shows the results.

TABLE 3

| | | | | | Resistance muta- | | |
|---|---|---|---|---|---|---|---|
| Sample | Exon 19 | | | | tion/activation | Biopsy tissue | Outline of EGFR- |
| No. | deleted | L858R | L861Q | T790M | mutation (%) | EGRF mutation | TKI treatment |
| 155 | 344 | 0 | 7 | 68 | 19.77 | Exon 19 deleted | EGFR-TKI sensitive |
| 152 | 15016 | 0 | 6 | 9 | NA | Exon 19 deleted | Before treatment with Iressa |
| 153 | 41922 | 0 | 16 | 33 | 0.08 | Exon 19 deleted | Case of progression after EGFR-TKI |
| 166 | 10 | 153 | 11 | 10 | NA | L858R | Suspicion of resistance |
| 154 | 1296 | 0 | 0 | 242 | 18.67 | Exon 19 deleted | Case of progression after EGFR-TKI |

The numerical values in Columns 2-5 show the number of sequences containing a mutation out of 100,000 determined sequences (the number of mutations in 100,000 was calculated for each item, and then the results were rounded off to integers).
Statistically Significance: 20 or more.
NA: Not applied. Not analyzed due to statistical insignificance.

As described above, it has become clear that the abundance ratio of resistance mutation can also be measured based on the number of molecules of resistance mutation and activation mutation when a next generation sequencer is used as in the case of BEAMing. In Sample No. 155, T790M was found though a clinical picture showed no resistance. In all the other cases, there was no discrepancy between clinical pictures and the state of T790M.

It goes without saying that the present invention is not limited to the abovementioned embodiment and can be modified in various manners without departing from the spirit and scope of the invention.

What is claimed is:

1. A method for assessing a progression of a clinical state of a malignant neoplasm in a subject who has been administered with a medicine for treating the malignant neoplasm, the method comprising:
(1) a step of determining a ratio of DNA molecules having an activation mutation that serves as an activation marker for the medicine to DNA molecules having a normal marker gene in DNA in the blood from the subject;
(2) a step of determining a ratio of DNA molecules having a resistance mutation that serves as a resistance marker for the medicine to DNA molecules having a normal marker gene in DNA in the blood from the subject; and
(3) a step of comparing a value obtained in the step (2) with a value obtained in the step (1).

2. The method according to claim 1, wherein:
the subject is a patient with non-small cell lung cancer;
the medicine is an EGFR inhibitor; and
the normal marker gene is a normal EGFR gene.

3. The method according to claim 2, wherein the resistance mutation is T790M in an EGFR gene.

4. The method according to claim 2, wherein the activation mutation is one or more mutations selected from ΔE746-A750, L858R, G719C, G719S and G719A in an EGFR gene.

5. The method according to claim 2, wherein the EGFR inhibitor is Gefitinib or Erlotinib.

6. The method according to claim 1, wherein the subject is a patient with chronic myeloid leukemia (CML), and the medicine is Imatinib.

7. The method according to claim 6, wherein the resistance mutation is T3151.

8. The method according to claim 6, wherein the activation mutation is bcr-abl.

9. The method according to claim 1, wherein the subject is a patient with lung cancer or pulmonary adenocarcinoma, and the medicine is an ALK inhibitor.

10. The method according to claim 9, wherein the resistance mutation is L1195M or C1156Y.

11. The method according to claim 9, wherein the activation mutation is EML4-ALK.

12. The method according to claim 9, wherein the ALK inhibitor is Crizotinib.

13. The method according to claim 1, wherein the step (1) and the step (2) are performed by using emulsion PCR.

14. A kit used for the method according to claim 1, the kit comprising a primer set used for detecting the activation mutation and a primer set used for detecting the resistance mutation.
